(19) United States
(12) Patent Application Publication
(10) Pub. No.: US 2007/0020318 A1
(43) Pub. Date: Jan. 25, 2007

(54) Hydrocolloid materials for use in wound healing

(76) Inventors: Derek W. Silcock, Skipton (GB); Michelle Delbono, Barnoldswick (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/548,231

(22) PCT Filed: Mar. 3, 2004
(86) PCT No.: PCT/GB04/00892

§ 371 Date: Aug. 18, 2006

Related U.S. Application Data
(60) Provisional application No. 60/458,383, filed on Mar. 31, 2003.

(30) Foreign Application Priority Data
Mar. 10, 2003 (GB) 0305454.1

(51) Int. Cl. A61L 15/00

(52) U.S. Cl. 424/445

(57) ABSTRACT

A wound dressing material comprising a low-moisture hydrocolloid matrix having oxidized cellulose distributed therein. For example, a matrix of dried sodium carboxymethylcellulose gel having fibers of oxidized regenerated cellulose dispersed therein. Also provided are the use of such materials in the treatment of wounds, and the manufacture of such materials by drying aqueous gels containing dispersed particles of oxidized cellulose.

The present invention relates to low-moisture gel compositions for use in wound dressings. The invention also relates to the manufacture of such low-moisture gel compositions, and to methods of wound treatment using such compositions.

EP-A-0918548 describes the use of oxidized celluloses, such as oxidized regenerated cellulose (ORC), for the treatment of wounds, in particular chronic wounds. Compositions are described that comprise milled ORC fibers dispersed in a 3% sodium carboxymethyl cellulose (NaCMC) aqueous hydrogel. Another example describes a composite film comprising ORC fibers dispersed in a plasticized collagen matrix. In other embodiments, the ORC fibers are dispersed in an aqueous collagen slurry and the mixture is freeze-dried to produce a collagen/ORC sponge having especially good properties for the treatment of chronic wounds. Such freeze-dried sponges are commercially available from Johnson & Johnson Medical Limited under the registered trade mark PROMOGRAN.

EP-A-0562862, EP-A-0562863 and EP-A-0562864 describe freeze-dried collagen sponges optionally having macroscopic substructures made of ORC embedded in the sponge. One embodiment consists of an ORC fabric laminated to a layer of collagen sponge and a layer of plasticized collagen film.

EP-A-0636378 describes medicated collagen films for use in the treatment of periodontal disease. The medicated collagen films may contain dispersed ORC fibers. The collagen matrix may contain up to 20% by weight, based on the weight of the composite of a plasticizer.

A need remains for further wound dressing materials containing ORC that can 30 provide advantages over ORC/CMC gels or collagen/ORC sponges in terms of cost, stability, ease of manufacture and/or other properties.

The present inventors have found that a stable, conformable wound dressing material can be made by dispersing solid ORC in an aqueous gel, followed by drying the gel to produce a stable, flexible hydrogel matrix material containing dispersed ORC.

Accordingly, in a first aspect the present invention provides a wound dressing material comprising a low-moisture hydrogel matrix having oxidized cellulose distributed therein.

The term “hydrogel matrix” refers to a continuous solid phase having the oxidized cellulose dissolved or embedded therein. The Hydrogel matrix is not a freeze-dried sponge material, and preferably it has an uncompressed density of at least about 0.2 g/cm³, more preferably at least about 0.4 g/cm^{3.}

The hydrogel matrix comprises one or more gel-forming hydrocolloids. The term “gel-forming hydrocolloid” refers to a polymeric material that absorbs water, for example from wound fluid, to form a coherent gel under physiological conditions of temperature and pH. Preferably, the hydrocolloid absorbs at least 100% w/w, more preferably at least 300% w/w of water on immersion at 25° C. for 24 hours. The matrix is preferably soluble in excess water, so as to release dispersed ORC into the wound in use. In other embodiments the matrix is water-swellable, but not water-soluble. In preferred embodiments of this type, the matrix may be formed of a hydrogel material that breaks down gradually in vivo so as to provide sustained release the oxidized cellulose into the wound. The term “hydrogel” in this context does not include gels or films comprising substantial amounts (e.g. more than 50% by weight) of gel-forming proteins or peptides such as collagen or gelatin. Preferably, the hydrogel material according to the present invention is substantially free of collagen and gelatin, and more preferably it is substantially free of other gel-forming proteins or peptides.

In certain embodiments the hydrogel matrix comprises a hydrocolloid material selected from the group consisting of modified celluloses, modified starches, alginates, plant gums, glycosaminoglycans, polyacrylates, polyurethanes, polymers of vinyl alcohols, vinyl esters, vinyl ethers and carboxy vinyl monomers, meth(acrylic) acid, acrylamide, N-vinyl pyrrolidone, acylamidopropane sulfonic acid, PLURONIC (Registered Trade Mark) (block polyethylene glycol, block polypropylene glycol) polystyrene-, maleic acid, NN-dimethylacrylamide diacetone acrylamide, acryloyl morpholine, and mixtures thereof.

In preferred embodiments the hydrocolloids comprise a naturally occurring or chemically modified polysaccharide, and preferably they consists essentially of one or more naturally occurring or chemically modified polysaccharides, or mixtures thereof. Suitable chemically modified polysaccharides are selected from the group consisting carboxymethyl cellulose gels, hydroxyethyl cellulose gels, hydroxy propyl methyl cellulose gels, chitosan, low-methoxy pectins, cross-linked dextran and starch-acrylonitrile graft copolymer, starch sodium polyacrylate, and mixtures thereof. Suitable natural polysaccharides include alginic acid and its salts, pectins, galactomannans such as xanthan gum or guar gum locust bean gum, gum karaya, gum arabic, hyaluronic acid and its salts, starches, and mixtures thereof. Suitably synthetic hydrocolloids include high molecular weight polyethylene glycols and polypropylene glycols, polymers of methyl vinyl ether and maleic acid and derivatives; polyvinyl pyrrolidone, polyethylene glycols, polypropylene glycols, metal and/or ammonium salts of polyacrylic acid and/or its copolymers, metal or ammonium salts of polystyrene sulfonic acid, and mixtures thereof.

Especially preferred are compositions in which the hydrocolloids comprise, or consist essentially of, a carboxymethyl cellulose salt.

Preferably, the oxidized cellulose is an oxidized regenerated cellulose. Preferably, the oxidized cellulose is a solid material forming a distinct solid phase embedded in the matrix. However, in certain embodiments the oxidized cellulose may be in the form of dissolved soluble fragments, as described in EP-A-0907664, the entire content of which is incorporated herein by reference.

Preferably, the oxidized cellulose is in the form of fibers or fiber fragments, more preferably in the form of a woven or non-woven fabric, or discrete fibers or fiber fragments dispersed in the matrix. Preferably, the fibers or fiber fragments are randomly dispersed in the matrix.

Preferably, the wound dressing material according to the present invention further comprises a plasticizer. Suitable plasticisers include glycerol, propylene glycol, polyethylene glycol, polypropylene glycol, sorbitol, other glycols and ether glycols such as mono- or diethers of polyalkylene glycol, mono- or diester polyalkylene glycols, polyethylene glycols glycolates, ethylene glycol, diethylene glycol, triethylene glycol, propylene glycol dipelargonate and polypropylene glycol glycerol, sorbitan esters, esters of citric and tartaric acid, imidazoline derived amphoteric surfactants, lactams, amides, polyamides, quaternary ammonium compounds, esters such phthalates, adipates, stearates, palmitates, sebacates, or myristates, and combinations thereof. diisopropyl adipate, phthalates and diethyl sebacate; hydrocarbons such as liquid paraffin; ethoxylated stearyl alcohol, glycerol esters, isopropyl myristate, isotridecyl myristate, ethyl laureate, N-methylpyrrolidone, ethyl oleate, oleic acid, isopropyl adipate, isopropyl palmitate, octyl palmitate, 1,3-butanediol and mixtures thereof. Preferably, the wound dressing material comprises from about 10% to about 80% by weight of the plasticizer, more preferably from about 15% to about 60% by weight of the plasticiser.

The hydrogel matrix is a low-moisture hydrogel material. It has been found that the low moisture content stabilizes the ORC in the matrix, and provides related advantages. The wound dressing material comprises from about 1% to about 50% by weight of water, preferably from about 5% to about 40% by weight of water, more preferably from about 10% to about 30% by weight of water.

Preferably, the wound dressing material according to the present invention comprises from about 1% to about 40% by weight of the oxidized regenerated cellulose, preferably from about 5% to about 25% by weight.

Preferably, the wound dressing material according to the present invention comprises from about 1% to about 80% by weight of the one or more hydrocolloids, more preferably from about 25% to about 60% by weight.

Preferably, the wound dressing material according to the present invention further comprises one or more therapeutic agents for promoting or enhancing wound healing. The one or more therapeutic agents may be any substance suitable for the treatment of wounds, but does not include the hydrogel used to form the matrix or the oxidized cellulose itself, both of which may independently promote wound healing. In certain embodiments the therapeutic agents are selected from the group consisting of antiseptics, antibiotics, analgesics, steroids and growth factors. Preferred therapeutic agents are the antimicrobials, in particular antibiotics and antiseptics such as colloidal silver, silver sulfadiazine, povidone iodine, chlorhexidine, and mixtures thereof. Preferably, the wound dressing material according to the present invention comprises from about 0.001% to about 10% by weight of the one or more therapeutic substances, more preferably from about 0.05% to about 2% by weight.

In a second aspect, the present invention provides a wound dressing comprising a wound dressing material according to the present invention.

For example, the material according to the present invention may be made in the form of a solid sheet (or layer) for application to a wound. Preferably, the sheet is from 0.1 to 2 mm thick. Preferably, the sheet has a dry basis weight of from about 10 to about 1000 g/m², more preferably from about 20 to about 200 g/m², and most preferably from about 40 to about 100 g/m². The sheet may be laminated to a solid polymer film, for example a perforated wound contacting film of ethylene methyl acrylate (EMA).

The sheet or layer of the material according to the present invention may be continuous or apertured. Typically, the apertures mal(e up from about 0.1% to about 50% of the area of the sheet (preferably of the wound facing area of the sheet) before swelling, more typically from about 1% to about 30% of the area of the sheet before swelling, preferably from about 10% to about 25%, and more preferably from about 10% to about 20%. of the area of the sheet before swelling.

It is an advantage of the present invention that the low moisture hydrogels have a conformable and slightly resilient feel that makes such sheets suitable for handling and application to a wound surface.

Preferably, the wound dressing comprises an absorbent layer and/or a backing layer in addition to the sheet of material according to the present invention, in which case the sheet is preferably the wound-facing top sheet of the dressing.

Preferably, the dressing further comprises a backing layer over the back face of the dressing sheet. The backing layer preferably provides a barrier to passage of microorganisms through the dressing and further preferably blocks the escape of wound fluid from the dressing. The backing layer may extend beyond at least one edge of the absorbent layer to provide an adhesive-coated margin adjacent to the said edge for adhering the dressing to a surface, such as to the skin of a patient adjacent to the wound being treated. An adhesive-coated margin may extend around all sides of the absorbent layer, so that the dressing is a so-called island dressing. However, it is not necessary for there to be any adhesive-coated margin.

Preferably, the backing layer is substantially liquid-impermeable. The backing sheet is preferably semipermeable. That is to say, the backing sheet is preferably permeable to water vapour, but not permeable to liquid water or wound exudate. Preferably, the backing sheet is also microorganism-impermeable. Suitable continuous conformable backing sheets will preferably have a moisture vapor transmission rate (MVTR) of the backing sheet alone of 300 to 5000 g/m²/24 hrs, preferably 500 to 2000 g/m²/24 hrs at 37.5° C. at 100% to 10% relative humidity difference. The backing sheet thickness is preferably in the range of 10 to 1000 micrometers, more preferably 100 to 500 micrometers.

Suitable polymers for forming the backing sheet include polyurethanes and poly alkoxyalkyl acrylates and methacrylates such as those disclosed in GB-A-1280631. Preferably, the backing sheet comprises a continuous layer of a high density blocked polyurethane foam that is predominantly closed-cell. A suitable backing sheet material is the polyurethane film available under the Registered Trade Mark ESTANE 5714F.

The adhesive (where present) layer should be moisture vapor transmitting and/or patterned to allow passage of water vapor therethrough. The adhesive layer is preferably a continuous moisture vapor transmitting, pressure-sensitive adhesive layer of the type conventionally used for island-type wound dressings, for example, a pressure sensitive adhesive based on acrylate ester copolymers, polyvinyl ethyl ether and polyurethane as described for example in GB-A-1280631. The basis weight of the adhesive layer is preferably 20 to 250 g/m², and more preferably 50 to 150 g/m². Polyurethane-based pressure sensitive adhesives are preferred.

Preferably, the adhesive layer extends outwardly from the absorbent layer and the envelope to form an adhesive-coated margin on the backing sheet around the adhesive layer as in a conventional island dressing.

The area of the optional absorbent layer is typically in the range of from 1 cm² to 200 cm², more preferably from 4 cm² to 100 cm².

The optional absorbent layer may be any of the layers conventionally used for absorbing wound fluids, serum or blood in the wound healing art, including gauzes, nonwoven fabrics, superabsorbents, hydrogels and mixtures thereof. Preferably, the absorbent layer comprises a layer of absorbent foam, such as an open celled hydrophilic polyurethane foam prepared in accordance with EP-A-0541391, the entire content of which is expressly incorporated herein by reference. In other embodiments, the absorbent layer may be a nonwoven fibrous web, for example a carded web of viscose staple fibers. The basis weight of the absorbent layer may be in the range of 50-500 g/m², such as 100-400 g/m². The uncompressed thickness of the absorbent layer may be in the range of from 0.5 mm to 10 mm, such as 1 mm to 4 mm. The free (uncompressed) liquid absorbency measured for physiological saline may be in the range of 5 to 30 g/g at 25°

Preferably, the wound dressing according to the invention is sterile and packaged in a microorganism-impermeable container.

In a third aspect, the present invention provides the use of a wound dressing material according to the present invention, for the preparation of a dressing for use in the treatment of wounds.

In a further aspect, the present invention provides a method of treatment of a wound in a mammal, comprising applying to the wound an effective amount of a wound dressing material according to the present invention.

Preferably, the wound is a chronic wound such as a venous ulcer, a diabetic ulcer or a decubitis ulcer.

In a further aspect, the present invention provides a method of making a wound dressing, comprising the steps of: providing an aqueous gel; immersing or dispersing an oxidized cellulose in the aqueous gel; followed by drying the gel to a moisture content of less than about 60% by weight.

Preferably, the method according to this aspect of the invention is adapted to the manufacture of a wound dressing material according to the present invention.

The aqueous gel preferably comprises at least about 90% w/w of water, more preferably at least about 95% w/w of water. The step of drying is preferably carried out under mild conditions. Preferably, the step of drying is applied without applying a vacuum, and preferably the step of drying is carried out at a temperature of from about 0 to about 100° C., more preferably from about 20° to about 80° C., and most preferably at from about 35° to about 65° C. The material may be shaped, for example by molding or extrusion, either before, after, or in the course of the drying step.

It will be appreciated that any features that are described as alternatives or preferred features in connection with any one of the above aspects of the invention are also likewise alternatives or preferred in relation to any other aspect of the invention.

Specific embodiments of the invention will now be described further, by way of example.

### EXAMPLE 1

Milled ORC fibers prepared as described in EP-A-1153622 (the entire content of which is incorporated herein by reference) were dispersed at a concentration of 2.17% w/w in KY Jelly (registered trade mark). KY Jelly is a commercial hydrogel manufactured by Johnson & Johnson. The exact formulation is as follows, in percentages by weight:

| | |
|---|---|
| Propylene glycol | 3.750 |
| Glycerine | 11.250 |
| Monosodium Dihydrogen | 0.875 |
| orthophosphate (buffer) | |
| Disodium hydrogen orthophosphate (buffer) | 0.045 |
| Methyl butex (preservative) | 0.100 |
| Propyl ester (preservative) | 0.040 |
| Hydroxyethyl cellulose | 2.133 |
| EDTA | 0.021 |
| Water | 81.792 |

The resulting gel was spread in a petri dish to a depth of 5 mm and dried in air immediately at 37° C. for 48 hours. The resulting dried hydrogel layer was flexible, conformable, and slightly elastic. The ORC in the hydrogel matrix appeared to be completely stable for at least six weeks at 37° C. in ambient atmosphere. That is to say, the ORC fibers when viewed under a microscope did not exhibit any swelling or dissolution.

### EXAMPLE 2

The procedure of Example 1 was repeated, but with replacement of the KY jelly with INTRASITE (Registered Trade Mark) gel produced by Smith & Nephew Healthcare Ltd. This is an aqueous gel which contains 2.3% of a modified CMC and 20% of propylene glycol.

The above embodiments have been described by of example only. Many other embodiments falling within the scope of the accompanying claims will be apparent to the skilled reader.

1. A wound dressing material comprising a low-moisture hydrogel matrix having oxidized cellulose distributed therein.

2. A wound dressing according to claim 1, wherein the oxidized cellulose is an oxidized regenerated cellulose.

3. A wound dressing according to claim 2, wherein the oxidized regenerated cellulose is in the form of a woven or non-woven fabric, or discrete fibers.

4. A wound dressing according to claim 1, wherein the hydrogel matrix comprises a hydrogel selected from the group consisting of modified celluloses, modified starches, alginates, plant gums, gelatins, glycosaminoglycans, polyacrylates, polyurethanes, and mixtures thereof.

5. A wound dressing according to claim 4, wherein the hydrogel is selected from the group consisting carboxymethyl cellulose salts, alginate salts, gelatins, hyaluronic acid and its salts, xanthan gum, guar gum, and mixtures thereof.

6. A wound dressing according to claim 1, wherein the wound dressing material further comprises a plasticizer.

7. A wound dressing according to claim 6, wherein the plasticizer is selected from the group consisting of glycerol, propylene glycol, polyethylene glycol, polypropylene glycol, sorbitol, other glycols and ether glycols such as mono- or diethers of polyalkylene glycol, mono- or diester polyalkylene glycols, polyethylene glycols glycolates, ethylene glycol, diethylene glycol, triethylene glycol, propylene glycol dipelargonate and polypropylene glycol glycerol, sorbitan esters, esters of citric and tartaric acid, imidazoline derived amphoteric surfactants, lactams, amides, polyamides, quaternary ammonium compounds, esters such phthalates, adipates, stearates, palmitates, sebacates, or myristates, and combinations thereof. diisopropyl adipate, phthalates and diethyl sebacate; hydrocarbons such as liquid paraffin; ethoxylated stearyl alcohol, glycerol esters, isopropyl myristate, isotridecyl myristate, ethyl laurate, N-methylpyrrolidone, ethyl oleate, oleic acid, isopropyl adipate, isopropyl palmitate, octyl palmitate, 1,3-butanediol and mixtures thereof.

8. A wound dressing according to claim 6, wherein the wound dressing material comprises from about 10% to about 80% by weight of the plasticizer.

9. A wound dressing material according to claim 1, wherein the wound dressing material comprises from about 1% to about 50% by weight of water.

10. A wound dressing material according to claim 1, wherein the material comprises from about 1% to about 40% by weight of the oxidized regenerated cellulose.

11. A wound dressing material according to claim 1, wherein the material comprises from about 1% to about 60% by weight of the one or more hydrocolloids.

12. A wound dressing material according to claim 1 further comprising one or more therapeutic agents.

13. A wound dressing comprising a wound dressing material according to claim 1.

14. A wound dressing according to claim 13, which is in the form of a sheet.

15. A wound dressing according to claim 13, which is sterile and packaged in a microorganism-impermeable container.

16. (Canceled)

17. (Canceled)

18. A method of making a wound dressing, comprising the steps of:
providing an aqueous gel;
immersing or dispersing an oxidized cellulose in the aqueous gel; followed by drying the gel to a moisture content of less than about 50% by weight.
